(19) United States
(12) Patent Application Publication
(10) Pub. No.: US 2024/0002782 A1
(43) Pub. Date: Jan. 4, 2024

(54) DENSITY-ADJUSTED LIQUIDS AND METHODS USING SAME

(71) Applicant: STEMCELL TECHNOLOGIES CANADA INC., Vancouver (CA)

(72) Inventors: Eric JERVIS, Vancouver (CA); Kyle HUKEZALIE, Vancouver (CA); Kylan WALSH, Vancouver (CA); Tia SOJONKY, Vancouver (CA); Karen MARKWICK, Vancouver (CA); Michael HIATT, Vancouver (CA)

(21) Appl. No.: 18/038,088

(22) PCT Filed: Nov. 25, 2021
(86) PCT No.: PCT/CA2021/051686

§ 371 Date: May 22, 2023

Related U.S. Application Data
(60) Provisional application No. 63/118,160, filed on Nov. 25, 2020.

(51) Int. Cl. C12N 5/00; C12N 5/074; C12N 5/0735

(52) U.S. Cl. CPC C12N 5/0018; C12N 5/0696; C12N 5/0606; C12N 2525/00

(57) ABSTRACT

Disclosed are liquids and methods for manipulating the localization and/or buoyancy of a population of particles, or one or more subpopulation thereof, within a volume of a liquid. The specific density of the liquids may be adjusted using specific gravity-modifying means depending on the application and/or on the average density of a population of particles, or one or more subpopulations thereof, within the liquid. For example, density-adjusted liquids may be used to bias a population of particles, or one or more subpopulations of particles, upward or downward within a volume of the liquid. As another example, density-adjusted liquids may be used to maintain the distribution of a population of neutrally buoyant particles, or one or more subpopulation thereof, within a volume of such liquid.

### RELATED APPLICATION

This application claims the benefit of U.S. Provisional Patent Application No. 63/118,160, filed Nov. 25, 2020, the entire content of which is hereby incorporated by reference in its entirety.

### FIELD OF THE DISCLOSURE

This disclosure relates to particles, or subpopulations thereof, immersed and/or suspended in a liquid, and more particularly to applications wherein a distribution or localization of such particles may be manipulated in the liquid medium.

### BACKGROUND OF THE DISCLOSURE

Whether in-use, during synthesis, or otherwise, particles are routinely immersed in a liquid environment. For example, nanoparticles, microparticles, or the like may be synthesized in a liquid medium or cells may be assayed or grown in a liquid medium. Numerous challenges arise because these operations are performed in a liquid medium, for example when synthesizing, assaying, sampling or recovering the particles.

Particles with a density higher than a density of liquid media will tend to settle within the liquid medium, which may limit exposure of their surface area to subunits required for synthesis or to reactants in the case of assays. Furthermore, a tendency of particles to settle results in a biased (e.g. non-homogeneous) distribution within the liquid, potentially impacting assaying or (successive) sampling operations. While settling of particles may in some cases facilitate their recovery, in other cases this tendency may not be desirable when, for example, a population of the particles is composed of one or more subpopulations of particles and only a certain subpopulation is of interest.

Means of redistributing particles in the liquid are known, but such means may be burdensome, time consuming, and/or interfere with the synthesis, assaying, sampling or recovery operations, or may in general negatively affect the health, quality, or well-being of the particles.

In the specific context of cell-based applications, cell culture processes are increasingly being adapted from monolayer conditions to suspension conditions. While suspension culture conditions may be more economical, in terms of output numbers of cells per unit volume of culture medium, numerous challenges stand in the way of widespread adoption. For example, cells grown in suspension will tend to settle to the bottom of the culture vessel or container. In order to realize the potential benefits of suspension culture the cells should be maintained in a suspended state, which usually is achieved by agitating the culture medium. However, agitating the culture medium will impact mass transfer across liquid-air or liquid-liquid interfaces of the particle-containing liquid, which may increase the oxygenation of the culture medium and/or impart shear forces on the growing culture of cells. Despite the commercialization of various cell culture bioreactors, at least the foregoing reasons have prevented the widespread adoption of suspension conditions in many cell culture processes.

Therefore, there is a need for reagents and processes that enable cost and time efficient synthesis (e.g. growth), assaying, sampling and/or recovery of particles, such as cells, when suspended in a liquid phase, while not negatively affecting their health, quality, or well-being.

### SUMMARY

The present disclosure relates to density-adjusted liquids and to methods using the density-adjusted liquids.

In one aspect of this disclosure are provided methods of manipulating the localization of a population of particles, or of one or more subpopulations thereof, within a volume/column of a liquid. Such methods may comprise: a) exposing the population of particles, or the one or more subpopulations thereof, to the liquid, the liquid supplemented with a specific gravity-modifying means and having a (relative) specific gravity that is not equal to unity; and b) influencing (and/or effecting a change in) buoyancy of the population of particles, or the one or more subpopulations thereof, within the volume/column of the liquid.

In one embodiment, the population of particles is a cell suspension, and subpopulations within the population may respectively comprise viable and dying or dead cells. Thus, liquids and or methods of this disclosure may be used in repeated sampling/aliquoting operations from a common volume/column of liquid, whether manual or automated, with a reduced risk of drawing dead or dying cells into daughter wells, while at the same time achieving a uniform or substantially uniform seeding density across the daughter wells seeded during such operations (in comparison to a liquid not supplemented with the specific gravity-modifying means and/or having a specific gravity equal to unity or substantially equal to unity).

In one embodiment, a (relative) specific gravity of the liquid is less than +/−0.5% from unity. In one embodiment, a (relative) specific gravity of the liquid is more than +/−0.5% from unity. In one embodiment, an average density of the population of particles, or the one or more subpopulations thereof, is different from the density of the liquid. In one embodiment, the specific density of the liquid is equal to unity.

In one embodiment, the methods may further comprise adjusting the specific gravity of the liquid before or after contacting the population of particles, or the one or more subpopulations thereof, with the liquid, by varying a concentration of the specific gravity-modifying means in the liquid.

In one embodiment, decreasing a concentration of the specific gravity-modifying means in the liquid involves diluting the liquid with additional liquid (e.g. the same or different liquid that includes a lower concentration of or no specific gravity-modifying means. In one embodiment, increasing a concentration of the specific-gravity-modifying means in the liquid involves adding additional of the same or a different specific gravity-modifying means to the liquid, whether in solid/powder form or as a concentrated liquid stock.

In one embodiment, the methods may further comprise applying an energy input to the liquid to distribute the population or subpopulation(s) of particles throughout the volume/column of the liquid. In one embodiment, the energy input is a turbulent mixing force. In one embodiment, the population of particles, or the one or more subpopulations thereof, are uniformly or homogenously distributed throughout the volume/column of the liquid.

In one embodiment, an increased specific gravity of the liquid corresponds to a decreased energy input to distribute the population or subpopulation(s) of particles within the volume/column of the liquid. In other words, the (relative) specific gravity (e.g. the density) of the liquid is inversely proportional to an energy input level for distributing the population of particles throughout the volume/column of the liquid.

In one embodiment, the methods may further comprise deactivating the energy input.

In one embodiment, (homogeneous or uniform) distribution of the population or subpopulation(s) of particles is maintained throughout the volume/column of the liquid after deactivating the energy input.

In one embodiment, the methods may further comprise removing a first aliquot of particles from the volume/column of the liquid. In one embodiment, the methods may further comprise removing a second aliquot of particles from the volume/column of the liquid, wherein a concentration of particles in the second aliquot is within about 80% or more of a concentration of particles in the first aliquot.

In one embodiment, an average density of the particles of the population, or the one or more subpopulations thereof, is the same as or within about +/−3% of the specific gravity (e.g. the density) of the liquid, and the population of particles, or the one or more subpopulations thereof, are neutrally buoyant within the volume/column of the liquid.

In one embodiment, the methods may further comprise biasing the population or subpopulation(s) of particles either upward from or downward toward a lower portion of the volume/column of the liquid.

In one embodiment, an average density of the particles of the population, or the one or more subpopulations thereof, is lower than the specific gravity (e.g. the density) of the liquid, and the population or subpopulation(s) is biased upward toward or to a gas-liquid interface of the volume/column of the liquid. In one embodiment, the population or subpopulation(s) of particles is sedimented within the volume/column of the liquid prior to contacting the population or subpopulation(s) of particles with the liquid and is biased upward within the volume/column of the liquid after coming into contact with the liquid.

In one embodiment, an average density of the particles of the population, or the one or more subpopulations thereof, is higher than the specific gravity (e.g. the density) of the liquid and the population or subpopulation(s) sediments within the volume/column of the liquid.

In one embodiment, the specific gravity of the liquid is between about 1.0 g/mL and 1.5 g/mL.

In one embodiment, the specific gravity of the liquid is more than or less than +/−3% from unity.

The specific gravity-modifying means can be any substance that is present within or may be added to the liquid and will have the effect to modify the (relative) specific gravity of the liquid. Examples of specific gravity-modifying means may include, whether in liquid or solid form, iodinated compounds (e.g. iodixanol, iopamidol, iohexol, or the like), perflourinated compounds, perfluorodecalin, salts (e.g. NaCl, tungsten salts, or the like), or density gradient medium such as may be sold under the brand names Lymphoprep™, Nycodenz™, Percoll™, or the like. In one embodiment, the specific gravity-modifying means is a specific gravity-modifying soluble solid or miscible solution. In one embodiment, the specific gravity-modifying soluble solid is iodixanol. In one embodiment, the specific gravity-modifying miscible solution is water miscible. In one embodiment, the specific gravity-modifying miscible solution is a non-ionic iodinated solution. In one embodiment, the non-ionic iodinated solution includes solubilized iodixanol.

In one embodiment, the population of particles, or the one or more subpopulations thereof, is a population or subpopulations of cells. In one embodiment, the population or subpopulation(s) of cells are single cells or aggregates of cells. In one embodiment, the population or subpopulation(s) of cells are stem cells, primary tissue derived cells or tissue fragments, or cell lines. In one embodiment, the stem cells are pluripotent stem cells, mesenchymal stem cells, hematopoietic stem or progenitor cells, neural stem or progenitor cells, or adult epithelial stem or progenitor cells. In one embodiment, the population or subpopulation(s) of cells are differentiated cells of mesoderm, ectoderm or endoderm lineages.

In one embodiment, the liquid is a cell culture medium.

In one embodiment, the methods may further comprise culturing and/or expanding the population or subpopulation(s) of cells within the volume/column of the liquid. In one embodiment, the growth and/or health and/or quality of the population or subpopulation(s) of cells is not adversely impacted in the liquid.

In another aspect of this disclosure are provided liquids for manipulating the localization and/or buoyancy of a population of particles, or one or more subpopulations thereof, comprising a diluent and a specific gravity-modifying means, wherein a specific gravity of the liquid is not equal to unity.

In one embodiment, the specific gravity-modifying means is a specific gravity-modifying soluble solid or miscible solution. In one embodiment, the specific gravity-modifying soluble solid is iodixanol. In one embodiment, the specific gravity-modifying miscible solution is water miscible. In one embodiment, the specific gravity-modifying miscible solution is a non-ionic iodinated solution. In one embodiment, the non-ionic iodinated solution includes solubilized iodixanol.

In one embodiment, the population of particles, or the one or more subpopulations thereof, is a population or subpopulation(s) of cells.

In one embodiment, the diluent is a buffer or a cell culture medium. In one embodiment, the cell culture medium is aqueous. In one embodiment, the cell culture medium is homogeneous. In one embodiment, a viscosity of the liquid is within 10% of water. In one embodiment, the viscosity is about 0.69 cP at 37° C.

In one embodiment, a specific gravity of the liquid is between about 1.0 g/mL and 1.5 g/mL.

In one embodiment, the liquid may further comprise one or more of a buffer, inorganic salts, trace elements, lipids, an albumin, growth factors, and cytokines.

In one embodiment, the specific gravity-modifying means does not sequester within the population or subpopulation(s) of cells.

In one embodiment, an average density of a population of particles, or of one or more subpopulations thereof, in the liquid is higher than the specific gravity of the liquid. In one embodiment, an average density of a population of particles, or of one or more subpopulations thereof, in the liquid is lower than the specific gravity of the liquid. In one embodiment, an average density of a population of particles, or of one or more subpopulations thereof, in the liquid is the same as or within +/−3% of the specific gravity of the liquid.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a plot of the relationship between density of medium used to culture cells, in this case PSC, and minimized RPM for suspending the cells throughout a density-adjusted medium of this disclosure.

FIG. 2 shows an image of PSC aggregates cultured at 20 rpm in 1.0 g/mL mTeSR™ 3D (A) or in mTeSR™ 3D density-adjusted to 1.048 g/mL (B). Images in (C) and (D) are respective magnifications of the images in (A) and (B).

FIG. 3 shows representative images of STIPS-F016 iPSC morphology after culturing for 3 days at 20 rpm in culture medium having a specific gravity of 1 g/mL (A), 1.024 g/mL (B), 1.032 g/mL (C), and 1.04 g/mL (D). Scale bar is 500 μm.

FIG. 4 shows a graph comparing daily fold expansion values for PSC aggregates cultured in either culture medium having a specific gravity of substantially 1 g/mL or in density adjusted culture medium have a specific gravity >1 g/mL. For 1 g/mL, mean=1.027, n=57. For >1 g/mL, mean=1.347, n=37. The lowest line in the box represents the 25th percentile, the middle line represents the 50th percentile (median) and the top most represents the 75th percentile. The “whiskers” are equal to 1.5×IQR (where IQR=interquartile range). p<0.0001 Student's T-test.

FIG. 5 shows effects of extended culture in density-adjusted medium on PSC. The cells of FIG. 2 grown in culture medium having a specific density greater than unity were further cultured in medium adjusted to 1.032 g/mL and assessed at passage 4 for daily fold expansion (A), percent viability (B), and expression of the pluripotency marker TRA-1-60 (C) relative to TRA-1-60 levels in a control culture grown in 2D in standard mTeSR1™ (D).

FIG. 6 shows the effects of density-adjusted media on sequential aliquots of PSC seeded from a common suspension of cells into respective wells of a multi-well plate. Aliquots of PSC into daughter wells from cells suspended in a density-adjusted medium (1.04 g/mL) had a more uniform seeding density (A) and percent viability (B) across all wells compared to cells aliquoted from a suspension in a control medium (1.0 g/mL).

FIG. 7 shows the results of a larger scale experiment than in FIG. 6, using a robotic liquid handler to seed aliquots of a source suspension of PSC into 48 daughter wells. (A) A histogram of seeding efficiency when the source cell suspension includes single PSC in either standard density medium (1.0 g/mL) or density-adjusted media (1.04 g/mL and 1.06 g/mL). (B) A histogram of the viability of the seeded cells of (A).

FIG. 8 shows the results of a larger scale experiment, than the findings shown in FIG. 6, using a robotic liquid handler to seed aliquots of a source suspension of PSC into 48 daughter wells. (A) A histogram of seeding efficiency when the source cell suspension includes clumps of PSC in either standard density medium (1.0 g/mL) or density-adjusted media (1.04 g/mL and 1.06 g/mL). (B) A histogram of seeding efficiency when the source cell suspension includes aggregates of PSC in either standard density medium (1.0 g/mL) or density-adjusted media (1.04 g/mL and 1.06 g/mL).

FIG. 9 shows the results of experiments for elucidating potential effects on monolayer-maintained PSC of repeated, punctuated exposure to density-adjusted media during passaging operations. When passaging a culture of PSC into daughter wells, dissociated PSC were resuspended and seeded for the first 48 hours of culture in density-adjusted medium. After 10 passages, the cumulative viable cell yield (A), percent viability (B), expression levels of pluripotency markers (C), genome integrity at assessed regions (D), karyotype (E), and the ability of PSC to differentiate to the three germ layers (F) among the cultured PSC was not significantly impacted using density-adjusted medium in comparison to medium having a specific gravity substantially equal to unity.

FIG. 10 shows the effects of using density-adjusted media to harvest WLS-1C PSC aggregates from the wells of a microwell plate. Aggregate loss was rescued using cell culture media adjusted for increasing density (1.1 g/mL, 1.2 g/mL and 1.3 g/mL) in comparison to a control medium having a standard density (1.0 g/mL).

FIG. 11 shows photographs of differential settling of aggregates within a volume/column of liquid in either standard (1 g/mL) culture medium, with aggregates shown by the white arrow (A) and in density-adjusted (1.08 g/mL) culture medium (B). A zoomed in photograph of (B) shows individual aggregates suspended homogeneously or more or less homogeneously throughout the volume/column of the liquid (C). Photograph of the residue after passing both cell suspensions through respective 37 um mesh filters, aggregates (black arrows) were present in the residue of standard (1 g/mL) culture medium (D).

FIG. 12 shows the effects of passaging PSC aggregates through a filter. After passaging the aggregates of FIG. 11 through a filter, for each condition the recovered cells in the filtrate were assessed for the number of viable cells recovered (A) and the % viability of such cells (B). A summary of the data is shown in (C).

### DETAILED DESCRIPTION

This disclosure relates to density-adjusted liquids and to methods using the density-adjusted liquids. More specifically, this disclosure relates to manipulating buoyancy and/or localization of particles, or one or more subpopulations thereof, in a volume/column of the density-adjusted liquids, and to applications whereby the buoyancy and/or localizations of particles, or one or more subpopulations thereof, is manipulated in a volume/column of a density-adjusted liquid to carry out a process.

Where used in this disclosure, the term “particles” refers to any biological or non-biological particles. Biological particles may include but are not limited to: cells, whether prokaryotic or eukaryotic, and aggregates thereof; subcellular components, such as organelles or extracellular vesicles; proteins; nucleic acids; or viruses. Non-biological particles may include but are not limited to: a particle comprising one or more metals and/or metalloids, or any other inorganic matter; or a particle that is not biological but comprises organic matter.

In certain embodiments, the particles may be a combination of a biological particle and non-biological particle. For example, the particles of the disclosure may comprise a cell complexed with one or more magnetic or magnetizable particle. The particles of this disclosure may range in mean diameter from the Angstrom level to millimeters. The particles of this disclosure should be capable of being suspended in embodiments of a density-adjusted liquid of this disclosure. As an illustrative example of the foregoing, the particles (or population of particles, or one or more subpopulations of the particles) should not be able to attach or are not attached to a wall of a vessel or containment means. In some embodiments, an average density of the particles, or the one or more subpopulations thereof, is about the same as or within +/−35%, or +/−30%, or +/−25%, or +/−20%, or +/−15%, or +/−10%, or +/−5%, or +/−3%, or +/−1%, or +/−0.5% of the specific gravity (or density) of the liquid in which they are contained. In some embodiments, an average density of the particles, or the one or more subpopulations thereof, is greater than the specific gravity (or density) of the liquid in which they are contained. In some embodiments, an average density of the particles, or the one or more subpopulations thereof, is lower than the specific gravity (or density) of the liquid in which they are contained. The density of the particles relative to the density of the liquid is an important consideration for the various applications disclosed herein.

Where used in this disclosure, the term “population of particles, or one or more subpopulations thereof”, including all variations of the term, refers to a set of particles within a volume/column of liquid that will respond similarly when exposed to a density-adjusted liquid. For example, each particle of such set of particles may have a certain minimum density or have a density that falls within a density range so that they will respond similarly when exposed to a density-adjusted medium. By way of further clarification only, the set of particles may have a density between 1 and 1.1 g/mL and the liquid may have a density of 1.3 g/mL (i.e. a specific gravity relative to water of 1.3 g/mL), thus the population of particles will rise upward within the volume/column of the liquid. A population of particles may comprise one or more subpopulations of particles. As an illustrative example, a population of particles may be a cell suspension, and the individual cells within the suspension may be categorized into one or more groups based on a shared characteristic (such as density, diameter, biomarker expression, cell cycle stage, etc, or any combination of the foregoing). In a more specific example, a cell suspension may include live cells and dying (or dead cells).

Where used in this disclosure, the term “specific gravity” (or in some cases relative specific gravity) refers to the ratio of the density of a liquid of this disclosure relative to the density of a reference liquid. By way of example, a specific gravity of a liquid of this disclosure that is not equal to unity means the density of a liquid of this disclosure is different from the density of a reference liquid. As a more specific example, a specific gravity of 1.3 for a liquid of this disclosure may equate to the liquid having a density of 1.3 g/mL, when water (1.0 g/mL) is used as the reference liquid. Thus, based on the (relative) specific gravity of a liquid of this disclosure, it will be predictable if a population of particles, or one or more subpopulations thereof, will sink, float, or localize in a specific compartment within a volume/column of the liquid. Where the liquid is water or a liquid having substantially the same density of water, the term specific gravity may be applicable. Where the liquid is not water and possesses a density substantially different from the density of water, the term relative specific gravity may be more applicable. Where the particles of a population, or one or more subpopulations thereof, neither float at or near the surface nor sink to or near the bottom of the volume/column of the liquid, they may be neutrally buoyant (i.e. the density of the population of particles, or one or more subpopulations thereof, is equivalent or sufficiently similar to that of the liquid). In some instances, it may be possible to achieve neutral buoyancy if the population (or subpopulation(s)) of particles have an average density within +/−3%, +/−2%, +/−1% or less of the density of the liquid. Particle(s) of a population, or subpopulation thereof, that are neutrally buoyant may in other words be described as being (homogeneously) distributed within the volume/column of the liquid or distributed throughout the volume/column of the liquid. The foregoing phrases may be interchanged in this disclosure, unless the context where the phrase is used would not reasonably permit such an interpretation.

Where used in this disclosure, the term “specific gravity-modifying means” refers to any substance that is present within or may be added to a liquid of this disclosure and will have the effect to modify the (relative) specific gravity of the liquid. In certain embodiments of this disclosure, it may be desirable or advantageous to utilize a specific gravity-modifying means that does not markedly increase the viscosity and/or osmolarity and/or osmolality of the liquid. The foregoing qualities may be particularly important where the particles are cells, or some other biological material. Examples of specific gravity-modifying means may include, whether in liquid or solid form, iodinated compounds (e.g. iodixanol, iopamidol, iohexol, or the like), perflourinated compounds, perfluorodecalin, salts (e.g. NaCl, tungsten salts, or the like), or density gradient medium such as may be sold under the brand names Lymphoprep™, Nycodenz™, Percoll™, or the like. If the population of particles are cells, then it will be important to select a density gradient medium that is not detrimental to their health, well-being or quality, while preferably also not markedly changing the osmolarity and/or osmolality and/or viscosity of the liquid after its density has been adjusted using the specific gravity-modifying means.

The (relative) specific gravity of the liquid may be modified positively or negatively away from or toward unity (i.e. unity being the value where the ratio of the density of the liquid and the density of a reference liquid, such as water, is equal to 1 or is substantially equal to 1, which may have the effect of neutral buoyancy on a population or one or more subpopulations of particles also having an average density the same or substantially the same as the density of the liquid). For example, if the (relative) specific gravity of a liquid is 1.3 g/mL it may be possible to adjust the (relative) specific gravity toward or to unity by adding an appropriate volume of a liquid having a lower concentration, or none, of the specific gravity-modifying means included therein. As another illustrative example, if the (relative) specific gravity of a liquid is 1.1 g/mL it may be possible to adjust the (relative) specific gravity further away from unity by adding an appropriate amount (whether in powder form or within a volume of liquid) of the specific gravity-modifying means thereto.

### Methods

In one aspect, the methods of this disclosure encompass those steps for manipulating the buoyancy of a particle, a population of particles, or one or more subpopulations of particles in a (volume or column of a) density-adjusted liquid, in order to manipulate their localization within the volume/column of the liquid. Specific examples or applications of the foregoing methods may include, but are not limited to: (i) distributing the population (or subpopulation) of particles throughout a volume/column of the liquid, such as at neutral buoyancy; (ii) biasing the population (or subpopulation) of particles upward within a volume/column of the liquid (such as to or near a gas- or air-liquid interface); (iii) biasing a sedimented population (or subpopulation) of particles upward within a volume/column of the liquid; (iv) biasing a population (or subpopulation) of particles downward within a volume/column of the liquid, or (v) fractionating subpopulations of a population of particles within a volume/column of a liquid (e.g. any combination of sedimenting, floating, and distributing throughout). In some embodiments, different populations of particles may be localized to different compartments/portions within the volume/column of the liquid. For example, a (sub-) population of particles may be biased upward within a volume/column of liquid (such as to a gas- or air-liquid interface) while a different (sub-)population of particles in the volume/column of the liquid may be biased downward, while still another (sub-)population or particles may be, optionally, neutrally buoyant or distributed throughout the volume/column of the liquid.

In one embodiment, a method of manipulating a population or one or more subpopulations of particles takes place in a volume/column of a liquid. The volume of the liquid will necessarily be contained in some type of containment means, and thus the three dimensional space of the liquid within the containment means may be referred to as a “column”. In some embodiments, the containment means may be a vessel having at least one openable end. In some embodiments, the containment means may be a vessel having one closed end and one closeable end, such as a flask, a graduated cylinder, or a centrifuge tube. In some embodiments, the containment means may be a vessel having at least two openable and closeable ends, such as a conduit or a length of tubing.

Steps of the method will include contacting the population or one or more subpopulations of particles with the liquid and effecting a change in buoyancy of or influencing the population or one or more subpopulations of particles within a volume/column of the liquid, in comparison to a liquid having a specific gravity of about unity. The change in buoyancy or influencing may be measured in comparison to the same operations performed in a liquid having a specific gravity equal to or substantially equal to unity.

The liquid may be any liquid that supports the population of particles. In embodiments where the particles are cells, the liquid may be any liquid that is supportive of the cells, such as a physiological saline solution or a culture medium appropriate for the type(s) of cells. One important characteristic of the liquid is its (relative) specific gravity, or a specific gravity range thereof. Thus, the liquid will comprise a diluent and a specific gravity-modifying means. In one embodiment, a concentration of the specific gravity-modifying means may be varied depending on the nature of the population or subpopulation(s) of particles. In one embodiment, a concentration of the specific gravity-modifying means may be varied depending on the stage of the method, as further described herein.

At some or all stages of the method, the liquid will include a specific gravity-modifying means. Thus, in some embodiments the liquid may have a (relative) specific gravity that is not equal to unity. In one embodiment, the liquid may have a (relative) specific gravity that is greater than unity. In other words, the density of the liquid would be greater than the density of a reference liquid, such as water. In one embodiment, the liquid may have a (relative) specific gravity that is lower than unity. In some embodiments, the specific gravity of the liquid is between about 0.5 and 5 g/mL. In some embodiments, the specific gravity of the liquid is between about 0.9 and 4 g/mL. In some embodiments, the specific gravity of the liquid is between about 1 and 2 g/mL.

In some embodiments, the specific gravity of the liquid is between about 1.0 g/mL and 1.5 g/mL. In some embodiments, the specific gravity of the liquid is between about 1.002 g/mL and 1.45 g/mL. In some embodiments, the specific gravity of the liquid is between about 1.004 g/mL and 1.4 g/mL. In some embodiments, the specific gravity of the liquid is between about 1.008 g/mL and 1.3 g/mL. In some embodiments, the specific gravity of the liquid is between about 1.01 g/mL and 1.2 g/mL. In some embodiments, the specific gravity of the liquid is between about 1.0 g/mL and 1.1 g/mL. In some embodiments, the specific gravity of the liquid is about 1.05 g/mL±0.03 g/mL. In some embodiments, a liquid having a specific gravity within any of the foregoing ranges may be used in one-step of the methods or processes described herein, while a liquid having a different specific gravity within any of the foregoing ranges may be used in a different step of the methods or processes described herein.

In some embodiments, the specific gravity-modifying means may be a specific gravity-modifying soluble solid or a specific gravity-modifying miscible solution. Where the specific gravity-modifying means is a soluble solid, it may be a non-ionic iodinated compound. In a specific embodiment, the specific gravity-modifying soluble solid is iodixanol. Where the specific gravity-modifying means is a specific gravity-modifying miscible solution, such solution may be miscible in the diluent (e.g. water or some other aqueous solution). In some embodiments, the specific gravity-modifying miscible solution is a solution including a non-ionic iodinated compound. In a specific embodiment, the non-ionic iodinated solution includes solubilized iodixanol.

In some embodiments, the liquid may possess a first specific gravity, which may subsequently be adjusted to a different specific gravity, such as by varying a concentration of the specific gravity-modifying means in the liquid. In one embodiment, the methods may include adjusting the specific gravity of the liquid before contacting the population of particles with the liquid, by varying a concentration of the specific gravity-modifying means in the liquid. In one embodiment, the methods may include adjusting the specific gravity of the liquid after contacting the population of particles with the liquid, by varying a concentration of the specific gravity-modifying means in the liquid. Adjustments to the (relative) specific gravity of liquids, such as those liquids used in the methods disclosed herein, may be accomplished as described herein (e.g. dilution or addition of more specific gravity-modifying means to the liquid).

In some embodiments, the liquid may be aqueous. In one embodiment the diluent may be water or water-based. In specific applications, the liquid may be a buffer or the liquid may be a cell culture medium. Where the liquid is a buffer or a cell culture medium, the liquid may include one or more of: buffering agent(s), inorganic salt(s), trace element(s), lipid(s), an albumin, growth factor(s), cytokine(s), or small molecule(s) (e.g. a Rho kinase inhibitor, such as Y-27632).

In some embodiments, the liquid is homogeneous (i.e. the liquid is in one phase). A homogeneous liquid may facilitate manipulating localization and/or a buoyancy of the plurality of particles in the volume/column of the liquid, such as when the specific gravity of the liquid is adjusted during a process. In most embodiments, the liquid is homogeneous with respect to at least the diluent and the specific gravity modifying means included therein.

Another important characteristic of the liquid may be its viscosity, or a viscosity range thereof. In some embodiments, the liquid may have a viscosity that is within about 1%, 2%, 5%, 10%, or 20% of the viscosity of water. In one embodiment, the viscosity of the liquid is about 0.69 cP at 37° C. In one embodiment, the specific gravity of a liquid may be adjusted with less than about 20% change to the viscosity of the liquid. In one embodiment, the specific gravity of a liquid may be adjusted with less than about 10% change to the viscosity of the liquid. In one embodiment, the specific gravity of a liquid may be adjusted with less than about 5% change to the viscosity of the liquid.

Another important characteristic of the liquid, in some embodiments, may be its osmolality or osmolality, or ranges thereof. In one embodiment, the specific gravity of a liquid may be adjusted with less than about 1%, 2%, 5%, 10%, or 20% change to the osmolality of the liquid.

In some embodiments, the methods may comprise applying an energy input to the liquid. The energy input may distribute the population of particles, or the one or more subpopulation thereof, within the volume/column of the liquid. Thus, any type of energy input is contemplated within this disclosure, including energy inputs that may be applied either directly to the liquid or indirectly to the liquid. In one embodiment, the energy input may be applied directly to the liquid, such as by an impeller or the like, or an input of pressurized (inert) gas. In one embodiment, the energy input may be applied indirectly to the liquid, such as by an orbital shaker or the like.

In one specific embodiment, the energy input may be a turbulent mixing force. In some embodiments, the energy input (e.g. the turbulent mixing force) is mediated via an impeller or mixing pendulum. In any such embodiment, the revolutions per minute (“rpm”) of the turbulent mixing force may be an important variable to optimize. For example, if the intention is to distribute the population (or one or more subpopulations) of particles homogeneously or substantially homogeneously within the volume/column of the liquid, too high of an rpm may undesirably concentrate the particles at the perimeter of the volume/column of the liquid and/or negatively impact the population of particles through turbulent mixing forces. Or, if the intention is to distribute the population of particles homogeneously or substantially homogeneously within the volume/column of the liquid, too low of an rpm may undesirably concentrate the particles, depending on the specific gravity of the liquid, at an upper or lower fraction of the volume/column of the liquid.

In one embodiment, the energy input, whether direct or indirect, may cause homogeneous distribution, or substantially homogeneous distribution, of the population of particles, or the one or more subpopulations thereof, within the volume/column of the liquid.

In one embodiment, an increased (relative) specific gravity of the liquid may correspond to a decreased energy input required to (homogeneously) distribute the population (or one or more subpopulations) of particles within the volume/column of the liquid. For example, where the (relative) specific gravity of the liquid is greater than unity, such as by ˜2, ˜3%, ˜4%, 5%, ˜10%, ˜15%, ˜20%, ˜25%, ˜30%, ˜35%, ˜40% or more, a relatively lower rpm, such as ˜10 rpm, ˜15 rpm, ˜20 rpm, ˜25 rpm, ˜30 rpm, ˜35 rpm, ˜40 rpm or more, may be sufficient to (homogeneously) distribute a population of particles within the volume/column of the liquid, such as particles having a density of about 1.0 g/mL. In contrast, a decreased (relative) specific gravity of the liquid may correspond to an increased energy input to (homogeneously) distribute the population (or one or more subpopulations) of particles within the volume/column of the liquid. For example, where the specific gravity of the liquid is slightly greater than unity, such as ˜5%, ˜4%, ˜3%, ˜2%, ˜1%, ˜0.9%, ˜0.8%, ˜0.7%, ˜0.6%, ˜0.5% or less, a relatively higher rpm, such as ˜35 rpm, ˜40 rpm, ˜45 rpm, ˜50 rpm or more, may be required to (homogeneously) distribute a population of particles in the volume/column of the liquid, such as particles having a density of about 1.0 g/mL. In this way, it may be seen that an energy input level (to distribute the population or one or more subpopulations of particles) is inversely proportional to the (relative) specific gravity of the liquid.

In embodiments where the population of particles, or a subset thereof, in the volume/column of the liquid are intended to be assayed, processed, sampled, and/or removed therefrom, it may be necessary to deactivate the energy input. However, this may be counterproductive if after deactivating the energy input the population or one or more subpopulations of particles rapidly equilibrate to a non-homogeneous or biased distribution within the volume/column of the liquid. In such cases, it may be desirable that the distribution (e.g homogeneous or substantially homogeneous) of the population or one or more subpopulations of particles is maintained within the volume/column of the liquid after deactivating the energy input, such as if the population of particles, or the one or more subpopulations thereof, are neutrally buoyant within the liquid. Thus, the (relative) specific gravity of the liquid can be carefully optimized for the application in question in order to achieve the desired distribution of the population or one or more subpopulations of particles within the volume/column of the liquid for the desired duration of time. In one embodiment, the population of particles, or the one or more subpopulations thereof, are neutrally buoyant within the column of the lipid. In one embodiment, such neutrally buoyant particles may need to be initially distributed within the volume/column of the liquid, such as by applying an energy input thereto, but will maintain the distribution throughout the liquid on deactivating the energy input.

For example, embodiments of the methods may comprise removing (or sampling or drawing) a first aliquot of particles from the volume/column of the liquid, after deactivating the energy input, while the population or one or more subpopulations of particles are homogenously distributed or substantially homogeneously distributed throughout the liquid. Where successive aliquots of particles may desirably be removed from the same volume of liquid, the homogeneous distribution (or substantially homogeneous distribution) of the population or one or more subpopulations of particles should be (more-or-less) maintained within the volume/column of the liquid for as long as is necessary to remove such successive aliquots. Thus, the methods may comprise removing a second aliquot of particles from the volume/column of the liquid, wherein a concentration of particles in the second (third, and so on) aliquot is about the same or within about 75%, 80%, 85%, 90%, 95% or more of a concentration of particles in the first and/or immediately preceding aliquot. Such sampling operations may be performed manually or by automation.

It may not always be the case that a (substantially) homogenous distribution of the population or one or more subpopulations of particles within the volume/column of the liquid is desired. Rather, in some applications a biased or non-random distribution of the population or one or more subpopulations of particles within the volume/column of the liquid may be desired. Obtaining a biased or non-random distribution of the population or one or more subpopulations of particles may be achieved after deactivating an energy input or may be achieved in the absence of an energy input altogether.

In one embodiment, it may be desirable to bias the population or one or more subpopulations of particles either upward or downward within the volume/column of the liquid, which may take place before, during, or after a process step has been taken.

Biasing the population or one or more subpopulations of particles upward within the volume/column of the liquid is achieved when the density of the liquid is greater than an average density of the particles of the population or one or more subpopulations of particles (for example in a liquid having a relative specific gravity greater than unity). By way of example, biasing the population or one or more subpopulations of particles upward from a lower compartment of the volume/column of the liquid may facilitate subsequent isolation of such population(s). Biasing the population or one or more subpopulations of particles upward from a lower portion or compartment of the volume/column of the liquid may facilitate fractionating a first population or subpopulation from a second population or subpopulation of particles. For example, where particles of the second population or subpopulation have a higher density than the particles of the first population or subpopulation of particles, then the first and second (sub)populations may localize to distinct positions within the volume/column of the liquid, depending on the specific gravity of the liquid. In this way, it may also be possible to fractionate still further populations or subpopulations of particles (e.g. third, fourth, or more) from the first and second populations and subpopulations. In one embodiment, a first (sub)population may localize at or near to the gas- or air-liquid interface of the volume/column of the liquid, a second (sub)population may localize at or near the bottom (i.e. sediment) of the volume/column of the liquid, and a third (sub)population may be intermediate the first and second (sub)populations within the volume/column of the liquid. In the foregoing embodiment, the density of the liquid would need to be higher than the average density of the first (sub)population, lower than the average density of the second (sub)population, and the same or about the same as the average density of the third (sub)population.

In one embodiment, a density of each particle of the population or one or more subpopulations of particles is lower than the specific gravity of the liquid, and the population or one or more subpopulations of particles is biased upward within the volume/column of the liquid, such as to or near a gas-liquid interface of the volume/column of the liquid. Biasing a population or one or more subpopulations of particles at or near the gas-liquid interface of the volume/column of the liquid may permit increasing the local dissolved gas concentration in the proximity of the particles at or near the gas-liquid interface.

In one embodiment, the population or one or more subpopulations of particles may initially be sedimented within the volume/column of the liquid, whether prior to exposing the population or one or more subpopulations of particles to the liquid or afterward (such as after deactivating the energy input, or where the average density of the population of particles, or one or more subpopulations thereof, is greater than the specific gravity of the liquid). Such sedimented population or one or more subpopulations of particles may be biased upward within the volume/column of the liquid upon adjusting the specific gravity of the liquid with a higher concentration of specific gravity-modifying means (e.g. to increase the (relative) specific gravity of the liquid). The specific gravity of the liquid may be adjusted by dissolving additional specific gravity-modifying means in the liquid, or by adding a concentrated stock solution of the specific gravity-modifying means to the liquid. In some embodiments, the liquid of the stock solution may be the same type of liquid as is already in contact with the population or one or more subpopulations of particles. In some embodiments, the foregoing may be less of a concern, and the liquids could be different. One embodiment where the nature of the first (e.g. the liquid already in contact with particles) and second (e.g. the stock liquid added to the first liquid to increase, or decrease, its (relative) specific gravity) liquids may matter is where the particles are or comprise a population or one or more subpopulations of cells, or other biological material. In such case, it may be desirable that the first and second liquids are the same in all or most respects except with respect to the concentration of the specific gravity-modifying means.

Biasing the population or one or more subpopulations of particles downward within the volume/column of the liquid may be achieved when the density of the liquid is lower than an average density of the particles of the population or one or more subpopulations of particles. By way of example, biasing the population or one or more subpopulations of particles downward toward a lower portion of the volume/column of the liquid may facilitate subsequent isolation of such population or subpopulation(s). As another example, biasing the population or one or more subpopulations of particles downward toward a lower portion of the volume/column of the liquid may facilitate fractionating such population or one or more subpopulations from a second population or one or more subpopulations of particles, such as where particles of the second population or one or more subpopulations thereof have a lower average density than the particles of the first population or one or more subpopulations thereof particles. As discussed above, such manipulation may also fractionate third, fourth and so on populations or one or more subpopulations of particles relative to one another within the volume/column of the liquid.

In one embodiment, an average density of the particles of the population, or one or more subpopulations thereof, is higher than the density of the liquid and such (sub)population(s) of particles sediments within the liquid.

Biasing the localization of the population of particles, or the one or more subpopulations thereof, within the volume/column of the liquid may be especially advantageous when it is desirable to fractionate the population or one or more subpopulations of particles from other particles also present in the liquid. Fractionating the population or one or more subpopulations of particles from other particles also present in the liquid may be done on the basis of a difference in the density of the particles of the population or one or more subpopulations of particles in comparison to other populations or one or more subpopulations particles and the liquid.

In a specific application of the foregoing methods, the population or one or more subpopulations of particles may be a population or one or more subpopulations of cells. In some embodiments, the population or one or more subpopulations of cells consist of a single type of cell. In some embodiments, the population or one or more subpopulations of cells comprise various types of cells. In some embodiment, the population or one or more subpopulations may comprise live and dying, and/or dead, cells. Live and dying, and/or dead, cells may be characterized by different densities, and the methods and liquids disclosed herein may be used to fractionate live from dying, and/or dead, cells. Such fractionated preparations may be subsequently used in successive sampling/drawing operations from a common volume/column of liquid, whereby particles distributed throughout the liquid may be reproducibly (in terms cell viability and/or seeding density) aliquoted into a plurality of daughter wells.

In some embodiments, the population or one or more subpopulations of cells may be single cells, clumps of several cells, or may be aggregates of cells. A suspension of single cells may be obtained using known digestive enzymes, such as trypsin or the like, to digest a monolayer culture of cells, or by trituration of a suspension culture of cells. Clumps of several cells may be obtained by incomplete digestion or trituration of, respectively, a monolayer culture of cells or a suspension culture of cells. In some embodiments, the population of cells may be seeded as single cells. In some embodiments, a population of cells seeded as single cells or clumps of cells may proliferate in the liquid to form into aggregates.

In some embodiments, the population or one or more subpopulations of cells may be primary tissue derived cells or tissue fragments.

In some embodiments, the population or one or more subpopulations of cells may be stem cells or progenitor cells. Non-limiting examples of stem cells or progenitor cells include pluripotent stem cells (whether naïve, embryonic or induced), mesenchymal stem cells, hematopoietic stem or progenitor cells, neural stem or progenitor cells, or adult epithelial stem or progenitor cells. In addition, the mesenchymal stem cells, hematopoietic stem or progenitor cells, neural stem or progenitor cells, or adult epithelial stem or progenitor cells may be pluripotent stem cell-derived.

In some embodiments, the population or one or more subpopulations of cells may be differentiated cells of mesoderm, ectoderm or endoderm lineages. Such differentiated cells of mesoderm, ectoderm, or endoderm lineages may be differentiated from appropriate precursor cells obtained from a subject, such as a patient or a donor. Or, such differentiated cells of mesoderm, ectoderm, or endoderm lineages may be differentiated from appropriate stem cells or progenitor cells.

In some embodiments, the population or one or more subpopulations of cells may be cell lines.

In embodiments where the population or one or more subpopulations of particles is optionally a population of cells, the liquid may be a buffer or a cell culture medium. In one embodiment, a specific gravity of liquids, including buffers or cell culture media may be equal to, approximately equal to (e.g. within less than +/−0.5% of unity), or not equal to unity (e.g. more than +/−0.5% of unity). In one embodiment, the specific gravity of the liquid is greater than unity. In one embodiment, the specific gravity of the liquid is lower than unity. The foregoing description of the specific gravity of the liquid relates to both scenarios where the population or one or more subpopulations of particles are biological in nature (e.g. cells), or where the population or one or more subpopulations are not biological in nature.

The cell culture medium may be any medium that supports the cells, such as a medium that supports the growth and/or proliferation of the population of cells. Thus, in such embodiments, the methods may further comprise culturing and/or expanding the population or one or more subpopulations of cells within the liquid. In other embodiments, the methods may further comprise expanding the population or one or more subpopulations of cells in a specific compartment or portion of the volume/column of the liquid (e.g. sedimented therein, distributed within/throughout the volume/column, or at a gas- or air-liquid interface thereof). In any such embodiment, it may be desirable that the growth or health of the population or one or more subpopulations of cells is not adversely impacted in the liquid. More specifically, it may be desirable that the growth or health of the population or one or more subpopulations of cells is not adversely impacted by the specific gravity-modifying means.

Potential detrimental effects on the health or well-being of the population or one or more subpopulations of cells (or other biological material) may be characterized in terms of morphology and/or function. In the particular case where the population or one or more subpopulations of particles are cells, their health and well-being may be measured in terms of morphology and/or function, and/or one or more of: population doublings or changes in cell number over time; viability or % viability; the expression or absence of expression of biomarkers; the capacity/ability to differentiate to downstream cell types; metabolic rates; or the presence/absence of mutations, such as medium- or large-scale chromosomal abnormalities, or the disproportionate accumulation (over multiple (i.e. 5-10) passages) of such abnormalities among the cells in the (sub)population(s). For specific cell types, such as PSC, various known/recurring chromosomal abnormalities may be used as a proxy for the health of the population. Also, PSC should be able to differentiate to any or all of the three germ layers, and beyond. Also, PSC not exposed to differentiation conditions should express certain pluripotency markers, such as TRA-1-60, OCT4, and others.

In some embodiments, it may be desirable to bias the population or one or more subpopulations of cells upward within the volume/column of the liquid, such as to or near to a gas- or air-liquid interface thereof. This may be accomplished by exposing the population or one or more subpopulations of cells to a liquid having a (relative) specific gravity higher than the average density of the population or one or more subpopulations of cells.

In different or related embodiments, the population or one or more subpopulations of cells may bud-off or detach from a culture of cells adhered to the closed bottom end of the container. Thus, such budded-off or detached population of cells may be selectively biased upward within the volume/column of the liquid or to the gas-liquid interface thereof, using appropriately formulated liquids (e.g. media), as described herein.

In embodiments where the population or one or more subpopulations of particles is a population or one or more subpopulations of cells, an energy input (e.g. a turbulent mixing force) may be applied to the liquid. Applying an energy input to the liquid may distribute the population or one or more subpopulations of cells (homogeneously, or substantially homogeneously) within or throughout the volume/column of the liquid. Depending on the nature of population or one or more subpopulations of cells, it may be important to optimize a level of the energy input to not adversely impact the population or one or more subpopulations of cells. Indeed, certain cell types are particularly sensitive to shear effects, such as pluripotent stem cells. Thus, if the energy input is a turbulent mixing force, too high of an rpm may result in shear effects negatively impacting the (health or well-being of the) population of cells, including shear-resistant clonal selection, failure to proliferate, differentiation, or even death. Or, conversely, too low of a turbulent mixing input may result in cell settling and/or the formation of large-scale aggregates, improper distribution of nutrients, or insufficient nutrients due particularly to the low solubility of oxygen and it's mass transfer dependence on air-liquid interface renewal and/or bubble distribution. In one embodiment, the rpm may be set to between about 5-100 rpm, or between about 10-50 rpm, or between about 20 and 45 rpm.

### Liquids

In another aspect, liquids of this disclosure encompass those compositions capable of manipulating the buoyancy of a population of particles exposed to such liquids. Any description above with respect to liquid(s) in the context of the methods of this disclosure, applies to the foregoing description of liquid(s) that may be commercialized and/or used decoupled from the particular methods described above. Where such liquids are used or commercialized, they may be sold in the form of a kit comprising at least a base liquid (or more than one base liquids), such as a culture medium or media, and a supplement comprising a specific gravity-modifying means. In one embodiment, the foregoing base liquid is provided having a (relative) specific gravity at or substantially at unity. In one embodiment, the base liquid (e.g. culture medium) is pre-formulated to include a specified concentration of specific gravity-modifying means, and the (relative) specific gravity of the liquid is not equal to unity. In one embodiment, such pre-formulated base liquid may be kitted with additional specific gravity-modifying means (whether in dry/powder/solid form or comprised in a stock liquid), and a user may optimize or adjust the concentration of the specific gravity-modifying means therein, as may be desired to carry out the operations described hereinabove. In some embodiment, any of the foregoing base liquids, and specific gravity-modifying supplements, may be further kitted with other supplements comprising one or more of growth factors, cytokines, proteins, peptides, small molecules, or other items useful for the culture of cells.

As described above, the liquid may be any liquid that supports the population or one or more subpopulations of particles. One important characteristic of the liquid is its (relative) specific gravity, or range thereof. Thus, the liquid will comprise a diluent and a specific gravity-modifying means.

A concentration of the specific gravity-modifying means may be varied depending on the nature of the population or one or more subpopulations of particles and/or the particular application for which the liquid is used. Thus, in some embodiments the liquid may have a (relative) specific gravity that is not equal to unity, but nevertheless a specific gravity that enables neutral buoyancy of the population or one or more subpopulations of particles (e.g. cells, wherein most cells have a density of 1.03 g/mL+/−0.01 g/mL). In some embodiments, the liquid may have a (relative) specific gravity that is not equal to unity, but nevertheless a specific gravity that enables the population or one or more subpopulations of particles (e.g. cells) to be biased upward or downward with the volume/column of the liquid.

In one embodiment, the liquid may have a (relative) specific gravity that is greater than unity. In other words, the density of the liquid would be greater than the density of a reference substance (e.g. water). In some embodiments, the (relative) specific gravity of the liquid is between about 1.0 g/mL and 1.5 g/mL. In some embodiments, the (relative) specific gravity of the liquid is between about 1.004 g/mL and 1.4 g/mL. In some embodiments, the (relative) specific gravity of the liquid is between about 1.006 g/mL and 1.2 g/mL. In some embodiments, the (relative) specific gravity of the liquid is between about 1.008 g/mL and 1.1 g/mL. In some embodiments, the (relative) specific gravity of the liquid is about 1.05 g/mL±0.03 g/mL.

In other embodiments, the (relative) specific gravity of the liquid may be below unity. In other words, the density of the liquid would be less than the density of a reference substance (e.g. water). In some embodiments, the (relative) specific gravity of the liquid is between about g/mL and 1.0 g/mL. In some embodiments, the (relative) specific gravity of the liquid is between about 0.5 g/mL and 0.6 g/mL. In some embodiments, the (relative) specific gravity of the liquid is between about 0.6 g/mL and 0.7 g/mL. In some embodiments, the (relative) specific gravity of the liquid is between about 0.7 g/mL and 0.8 g/mL. In some embodiments, the (relative) specific gravity of the liquid is between about 0.8 g/mL and 0.9 g/mL. In some embodiments, the (relative) specific gravity of the liquid is between about 0.9 g/mL and 1.0 g/mL.

In one embodiment, an average density of a population of particles, or of one or more subpopulations thereof, in the liquid is higher than a specific gravity of the liquid. In one embodiment, an average density of a population of particles, or of one or more subpopulations thereof, in the liquid is lower than a specific gravity of the liquid. In one embodiment, an average density of a population of particles, or of one or more subpopulations thereof, in the liquid is the same as or within +/−3% of a specific gravity of the liquid.

In some embodiments, the specific gravity-modifying means may be a specific gravity-modifying soluble solid or a specific gravity-modifying miscible solution. Where the specific gravity-modifying means is a soluble solid, it may be a non-ionic iodinated compound. In a specific embodiment, the specific gravity-modifying soluble solid is iodixanol. Where the specific gravity-modifying means is a specific gravity-modifying miscible solution, such solution may be miscible in the diluent (e.g. water or some other aqueous solution). In some embodiments, the specific gravity-modifying miscible solution is a solution including a non-ionic iodinated compound. In a specific embodiment, the non-ionic iodinated solution includes solubilized iodixanol.

In some embodiments, the liquid may possess a first specific gravity, which may subsequently be adjusted to a different specific gravity, such as by varying a concentration of the specific gravity-modifying means. In one embodiment, the liquid may have a first specific gravity, which may be adjusted to a different specific gravity before contacting the population of particles with the liquid, by varying a concentration of the specific gravity-modifying means in the liquid. In one embodiment, the liquid may have a first specific gravity, which may be adjusted to a different specific gravity after contacting the population of particles with the liquid, by varying a concentration of the specific gravity-modifying means in the liquid. The concentration of the specific gravity-modifying means in the liquid may be adjusted as described elsewhere in this disclosure.

In some embodiments, the liquid may be aqueous. In one embodiment, the diluent may be water.

In some embodiments, the liquid is homogeneous (i.e. the liquid is in one phase). A homogeneous liquid may help to resolve changes in buoyancy of the plurality of particles, such as when the specific gravity of the liquid is adjusted during a process. In any event, the liquid should be homogeneous with respect to the diluent and the specific gravity modifying means.

Another important characteristic of the liquid may be its viscosity, or range thereof. In some embodiments, the liquid may have a viscosity that is within about 1%, 2%, 5%, 10%, or 20% of the viscosity of water. In one embodiment, the viscosity of the liquid is about 0.69 cP at 37° C. In some instances, the specific gravity of a liquid may be adjusted with less than about a 20%, 15%, 10%, 5%, 2% or 1% change to the viscosity of the liquid.

Another important characteristic of the liquid, in some embodiments, may be its osmolality or osmolality, or ranges thereof. In some instances, the specific gravity of a liquid may be adjusted with less than about a 20%, 10%, 5% or 2% change to the osmolality of the liquid.

In a specific application, the population or one or more subpopulations of particles is a population or one or more subpopulations of cells. In some embodiments, the population or one or more subpopulations of cells may be primary tissue derived cells or tissue fragments.

In some embodiments, the population or one or more subpopulations of cells may be stem cells or progenitor cells. Non-limiting examples of stem cells or progenitor cells include pluripotent stem cells (whether naïve, embryonic or induced), mesenchymal stem cells, hematopoietic stem or progenitor cells, neural stem or progenitor cells, or adult epithelial stem or progenitor cells. In addition, the mesenchymal stem cells, hematopoietic stem or progenitor cells, neural stem or progenitor cells, or adult epithelial stem or progenitor cells may be pluripotent stem cell-derived.

In some embodiments, the population or one or more subpopulations of cells may be differentiated cells of mesoderm, ectoderm or endoderm lineages. Such differentiated cells of mesoderm, ectoderm, or endoderm lineages may be differentiated from appropriate precursor cells obtained from a subject, such as a patient or a donor. Or such differentiated cells of mesoderm, ectoderm, or endoderm lineages may be differentiated from appropriate stem cells or progenitor cells.

In some embodiments, the population or one or more subpopulations of cells may be cell lines.

Thus, the liquid should support the population or one or more subpopulations of cells. A liquid that is supportive of a population or one or more subpopulations of cells should not have detrimental impacts upon the population of cells. Detrimental effects upon cells (e.g. health and/or well-being) may be measured as described above, or in any other way known to the skilled person. Further, a liquid that is supportive of a population or one or more subpopulations of cells should maintain the cells in a homeostasis or may support their maintenance and/or growth and/or proliferation and/or differentiation. Where the liquid maintains the cells in a homeostasis, it may not be suitable for long-term exposure to the population or one or more subpopulations of cells in terms of its composition. Nevertheless, a liquid supportive of a population or one or more subpopulations of cells should be physiological in terms of its composition (e.g. temperature, pH, salt concentration, etc.). By way of non-limiting example, the liquids of such application may be buffers or may be cell culture media. Where the liquid is a buffer or a cell culture medium, the liquid may include one or more of: buffering agent(s), inorganic salt(s), trace element(s), lipid(s), an albumin, growth factor(s), cytokine(s), or small molecule(s) (e.g. a Rho kinase inhibitor).

In applications where the population or one or more subpopulations of particles is a population of cells, the specific-gravity modifying means of the liquid should not sequester within the population or one or more subpopulations of cells.

The following non-limiting examples are illustrative of the present disclosure.

### EXAMPLES

### Example 1: Maintenance Culture of PSC in Monolayer or in Suspension

Pluripotent stem cells were maintained in either mTeSR™1, mTeSR™3D or TeSR™-E8™ 3D in accordance with the manufacturer's suggested protocols.

For cells maintained in a monolayer, cultures were grown on Matrigel™ (Corning) or Vitronectin (STEMCELL Technologies) and passaged upon reaching a confluency of about 50-80%. To passage the cells, media was aspirated, and the cells were washed with PBS. Either Gentle Cell Dissociation Reagent (STEMCELL Technologies), Accutase™ (STEMCELL Technologies) or ReLeSR (STEMCELL Technologies) was used to enzymatically digest the colonies. In the case of ReLeSR, undifferentiated PSC colonies could be preferentially detached from the culture surface. Detached PSC colonies were triturated to break them into clumps or single cells.

For cells maintained in suspension, cultures were grown in either TeSR™ E8™ 3D or mTeSR™ 3D (both STEMCELL Technologies) according to the manufacturers recommended protocols. Cells were grown in suspension as aggregates of undifferentiated cells (i.e. maintained in suspension) and passaged upon reaching a desired density (e.g. 8×10⁵ cells/mL), usually obtainable after 3-4 days when cultured as described herein. The aggregates grown in suspension were collected by centrifugation and washed using PBS. The aggregates were dissociated using either Gentle Cell Dissociation Reagent (STEMCELL Technologies) or Accutase™ (STEMCELL Technologies) in combination with trituration to break them into clumps or single cells.

### Example 2: Relationship of PSC Distribution and Minimum Rpm for Suspending the PSC

STIPS-M001 PSC were maintained as described in Example 1. At the time of passaging, clumps of cells were seeded into 50 mL TeSR-E8-3D (STEMCELL Technologies) in a single pendulum spinner flask (Integra Biosciences). The medium was supplemented with 10 μM ROCK inhibitor (STEMCELL Technologies). The cells were cultured for 3 days at rpm until they were large enough to see without a microscope and fed daily using TeSR-E8 3D Feed Supplement (STEMCELL Technologies) according to the manufacturer's suggested protocol.

Once the PSC aggregates reached the indicated size, they were seeded into various density-adjusted media (as prepared according to the table below) to assess the lower bounds of an rpm needed to approximately homogenously distribute the cells within the media tested (also as shown in the table below).

| | | | | | | |
|---|---|---|---|---|---|---|
| Density (g/mL) | 1.0000 | 1.0096 | 1.0192 | 1.0288 | 1.0384 | 1.0480 |
| Volume Iodinated Solution | 0 | 1.5 | 3 | 4.5 | 6 | 7.5 |
| (1.32 g/mL) (mL) | | | | | | |
| Volume Complete Medium | 50 | 48.5 | 47 | 45.5 | 44 | 42.5 |
| Min. rpm | 32.5 | 30 | 27.5 | 22.5 | 20 | 15 |

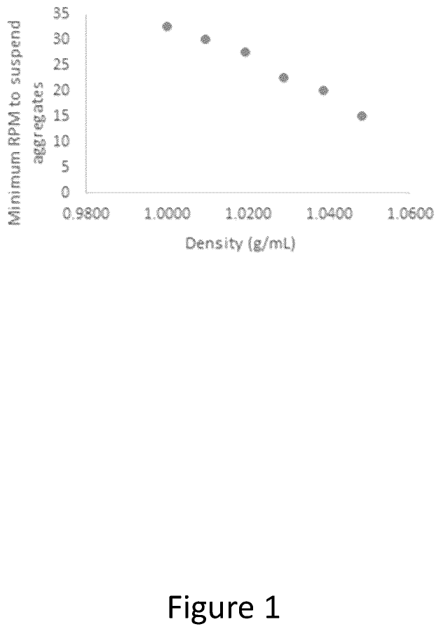

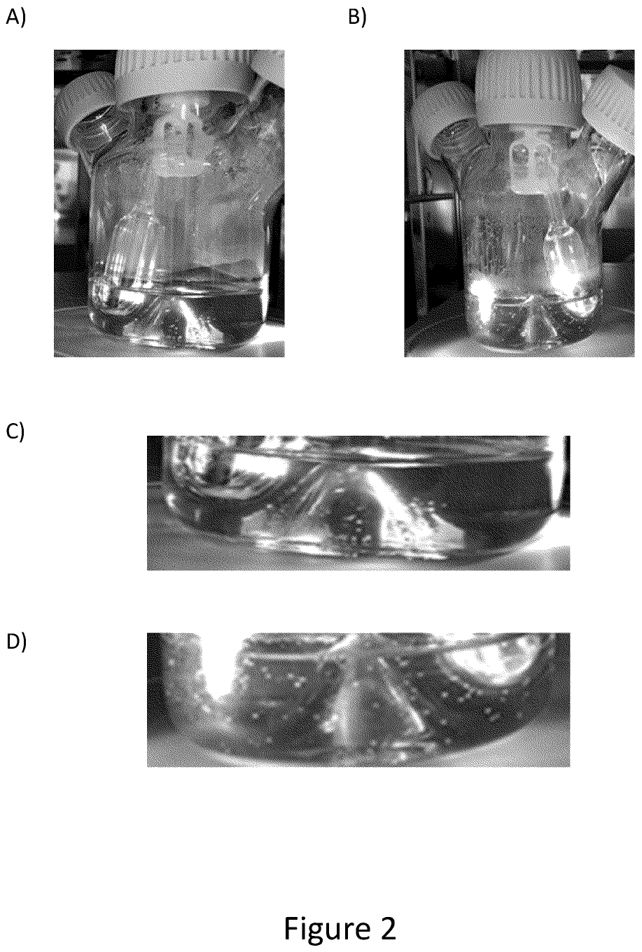

FIG. 1 shows the relationship between the density of culture medium and the rpm required to suspend aggregates of PSC in the culture medium. As the density of the medium is increased, less rpm is needed to distribute the PSC aggregates throughout the liquid medium. FIG. 2 shows a representative image of PSC aggregates suspended under the influence of a 20 rpm mixing speed in either a medium having a control density (1.0 g/mL) (FIG. 2A, and magnified in FIG. 2C) or in density-adjusted medium (1.048 g/mL) (FIG. 2B, and magnified in FIG. 2D). In the control culture, the aggregates are sedimented about the indent in the underside of the spinner flask, while in the density-adjusted culture the aggregates are well distributed (FIGS. 2C and 2D).

### Example 3: Scale-Up of PSC Suspension Cultures in Density-Adjusted Media

STIPS-F016 pluripotent stem cells were cultured and harvested as described in Example 1, and 1×10⁵ cells/mL were seeded into a 100 mL single pendulum spinner flask (Integra Biosciences), a 250 mL dual pendulum spinner flask (Integra Biosciences), and a 500 mL dual pendulum spinner flask (Integra Biosciences). In cultures under control conditions, each of the foregoing vessels was filled to half its volume with the complete media described in Example 2 (e.g. a non-density adjusted medium), and the cultures were fed, also as described in that Example. In parallel, each size of vessel was respectively filled to half its volume with media having its density adjusted (as described in Example 2) to 1.024 g/mL, 1.032 g/mL, and 1.04 g/mL. The cultures were passaged every 3-4 days and carried for 3 passages at 20 rpm (for the 100 mL flask) or 25 rpm (for the 250 mL and 500 mL flasks).

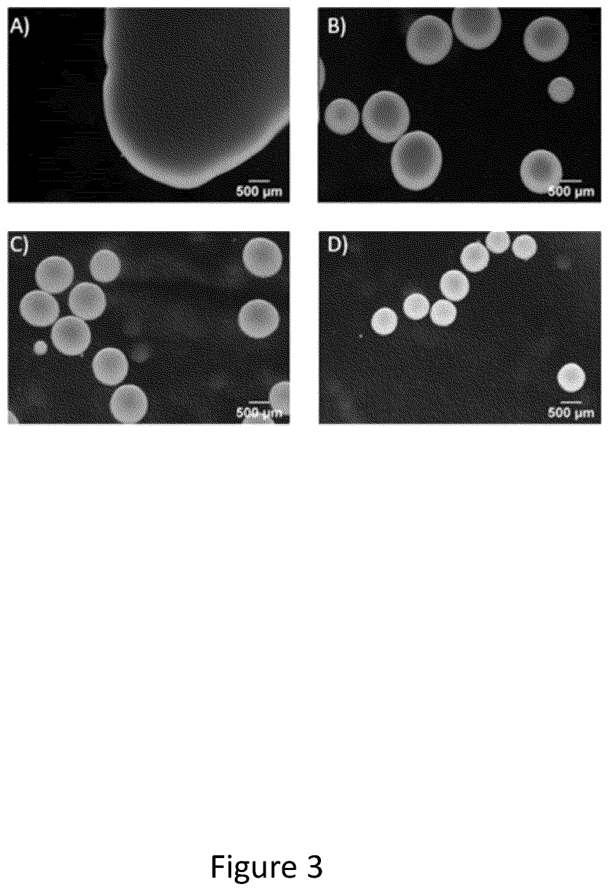

Microscope images were taken daily, and representative images for the 100 mL flask are shown in FIG. 3A-D. Since the control culture was subjected to half the recommended mixing speed, a single aggregate of PSC formed at the bottom of the flask, as expected (FIG. 3A). In contrast, as the density of the culture medium increased, a plurality of distinct aggregates could be observed within the culture medium (FIG. 3B-D). In general, as the density of the medium increased the size of the aggregates decreased.

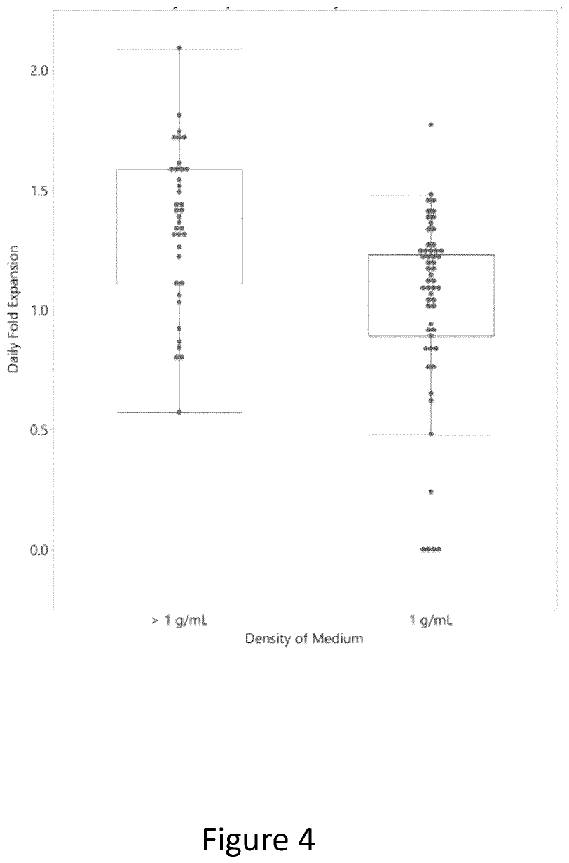

The daily fold expansion of PSC lines grown in suspension in either density-adjusted culture media (e.g. >1 g/mL) or in standard culture medium (e.g. 1 g/mL) was assessed (FIG. 4). The plot in FIG. 4 represents an amalgamation of all data points obtained from experiments growing STIPS-F016, STIPS-M001, WLS-1C, H9 and H1 PSC lines in suspension in either standard culture medium (n=57) or density-adjusted culture media (n=37). The data show that suspension culture in density-adjusted media achieves a higher daily fold expansion (mean=1.347) in comparison to standard culture medium (mean=1.027).

### Example 4: Extended Exposure of PSC to Density-Adjusted Medium

The cells from Example 2 were passaged (as described in Example 1) and seeded at 1×10⁵ cells/mL in a 100 mL single pendulum spinner flask (Integra). The cells were cultured and fed using the same culture media as described in Example 2 except the density was adjusted to 1.032 g/mL (5 mL iodixanol in 45 mL complete medium).

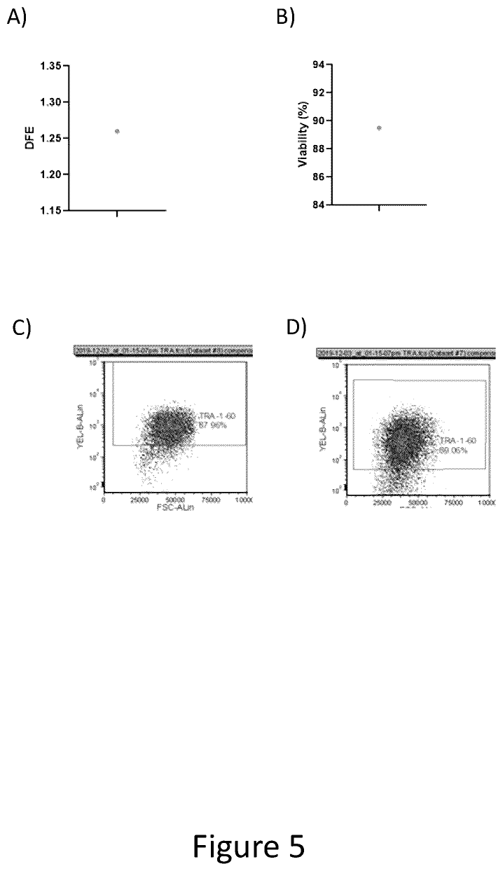

After 4 days in culture, the aggregates were harvested and dissociated using Accutase™ (STEMCELL Technologies) according to the suggested protocol. The dissociated cells were analyzed for daily fold expansion (FIG. 5A), percent viability (FIG. 5B), and for the expression of a pluripotency marker by flow cytometry (FIGS. 5C and 5D). PSC grown in culture medium having a density of 1.032 g/mL showed a daily fold expansion above 1.25 (FIG. 5A) and ˜90% of the cells were viable (FIG. 5B).

To assess the pluripotency of the PSC cultured in density-adjusted medium, the cells were stained with a PE anti-human TRA-1-60 R antibody (BioLegend). TRA-1-60 expression was analyzed by flow cytometry (Guava EasyCyte 12HT flow cytometer (Millipore)). Pluripotency of the cells was demonstrated, as roughly 89% of the analyzed cells expressed TRA-1-60 (FIG. 5D), comparable to expression levels (˜88%) of the same marker among F016 PSCs cultured under control conditions in 2D in mTeSR1 (FIG. 5C).

### Example 5: Reduced Seeding Density Variability in Manually Seeded Daughter Wells Using Density-Adjusted Media

F016 PSC (iPSC) were maintained and dissociated as described in Example 1. The dissociated clumps of pluripotent stem cells were resuspended at 5.00×10⁵ cells/mL in either control medium (1.0 g/mL) or density-adjusted medium (1.04 g/mL) from which successive aliquots were drawn and seeded into daughter wells.

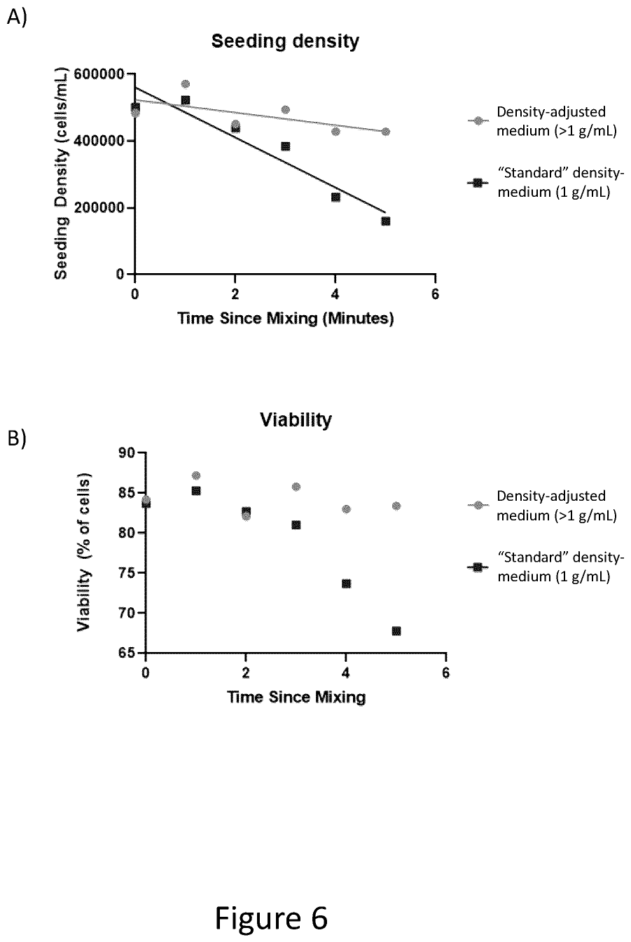

In this early experiment, where dissociated PSCs were manually sampled/aliquoted and seeded into daughter wells, no significant decrease in seeding density is observed among aliquoted clumps drawn in one-minute intervals from a cell suspension in density-adjusted medium. In contrast, clumps that were sampled from a cell suspension in control medium (i.e. “Standard” density medium” exhibited a decrease in seeding density dependent upon the amount of time that elapsed between successive sampling/aliquoting operations (FIG. 6A). Thus, the use of density-adjusted medium to prepare a suspension of PSC for subsequent seeding of daughter wells obviates the time-consuming step of thoroughly mixing a suspension of cells between successive operations of sampling and seeding multiple aliquots. Interestingly, it was also observed for those cells successively sampled/drawn from a suspension in density-adjusted medium and seeded into daughter wells exhibited increased viability in comparison to the same operation using cells suspended in control medium (i.e. “Standard” density medium) (FIG. 6B). Without being bound by theory, it is possible that there exists a density difference between live and dead cells, wherein live cells settle more rapidly than dead cells in the suspension of cells.

### Example 6: Reduced Seeding Density Variability in Robotically Seeded Daughter Wells Using Density-Adjusted Media

The experiments described in Example 5 and reported in FIG. 6 were extended to a larger scale experiment using a MicroLab STARlet liquid handling robot (Hamilton). Briefly, PSC maintained in a monolayer were dissociated into single cells using Accutase™ or into clumps using ReLeSR, as described in Example 1. In addition, PSC were formed into aggregates using an AggreWell™I 400 microwell device (STEMCELL Technologies) by depositing about 500 H1 cells per microwell and incubating the cells for 24 hours in mTeSR™ 1.

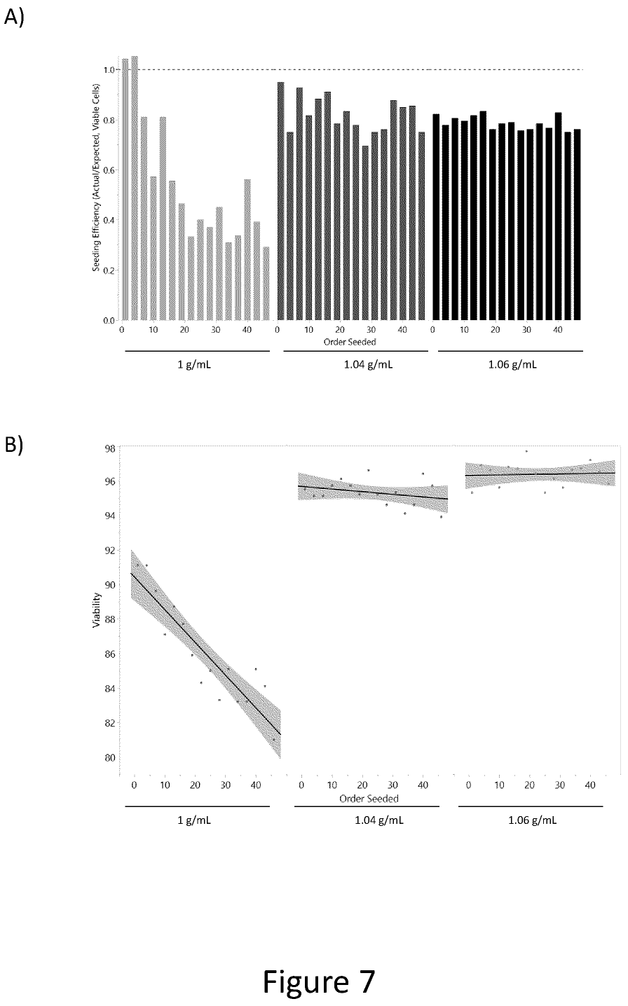

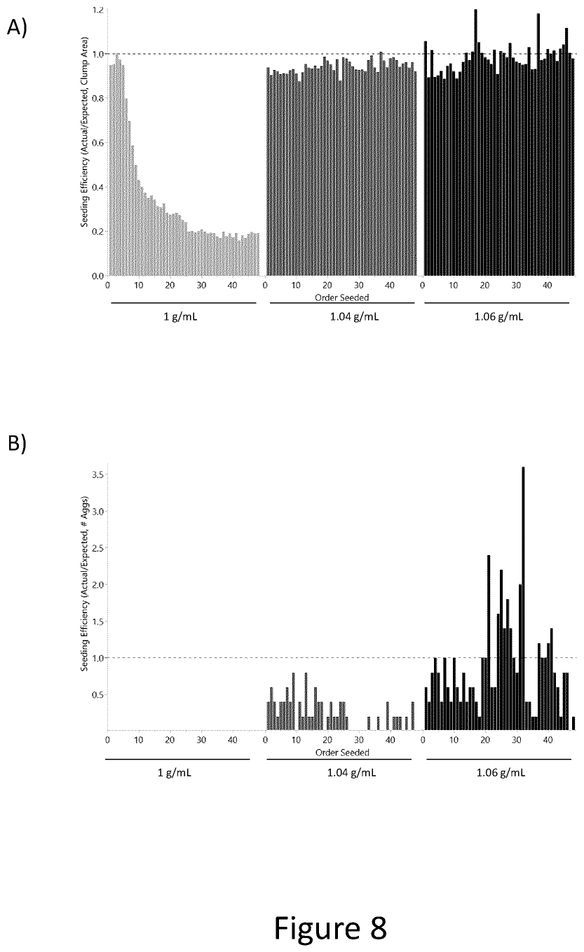

Three single cell suspensions of 2×10⁶ cells/mL were prepared in either control medium (1.0 g/mL) or in density-adjusted media (1.04 g/mL or 1.06 g/mL). The liquid handler seeded 48 daughter wells from each single cell suspension. The density-adjusted media yielded more consistent seeding efficiencies in comparison to the control medium (FIG. 7A). Further, the percent viability among the cells seeded into daughter wells from cell suspensions in density-adjusted was improved (FIG. 7B), similar to the observations reported in FIG. 6. The same trend of more consistent seeding density among daughter wells was also observed when aliquots of clumps of PSC (FIG. 8A) and aggregates of PSC (FIG. 8B) are taken from source a suspension (of clumps or aggregates) in density-adjusted media in comparison to aliquots taken from a source suspension in control medium.

### Example 7: Effects on Monolayer-Maintained PSC of Punctuated Exposure to Density-Adjusted Media During Passaging Operations

H9 (ES cells) and M001 (iPS cells) PSC were maintained for 10 passages in 2D on Matrigel in mTeSR1, essentially as described in Example 1. On day 7 of each passage, cultures were dissociated using ReLeSR and PSC clumps were counted and resuspended in density-adjusted culture medium (1.06 g/mL). The cell suspension was seeded at 2×10⁴ viable H9 cells/well and 1.5×10⁴ viable M001 cells/well into separate wells in media having either a final density of 1.0 g/mL, 1.005 g/mL, or 1.01 g/mL. On day 2 (i.e. after ˜48 hours of iodixanol exposure), the media was changed and all conditions were fed with standard mTeSR™1 culture medium (at 1.00 g/mL) for the remainder of the passage (days 2-6).

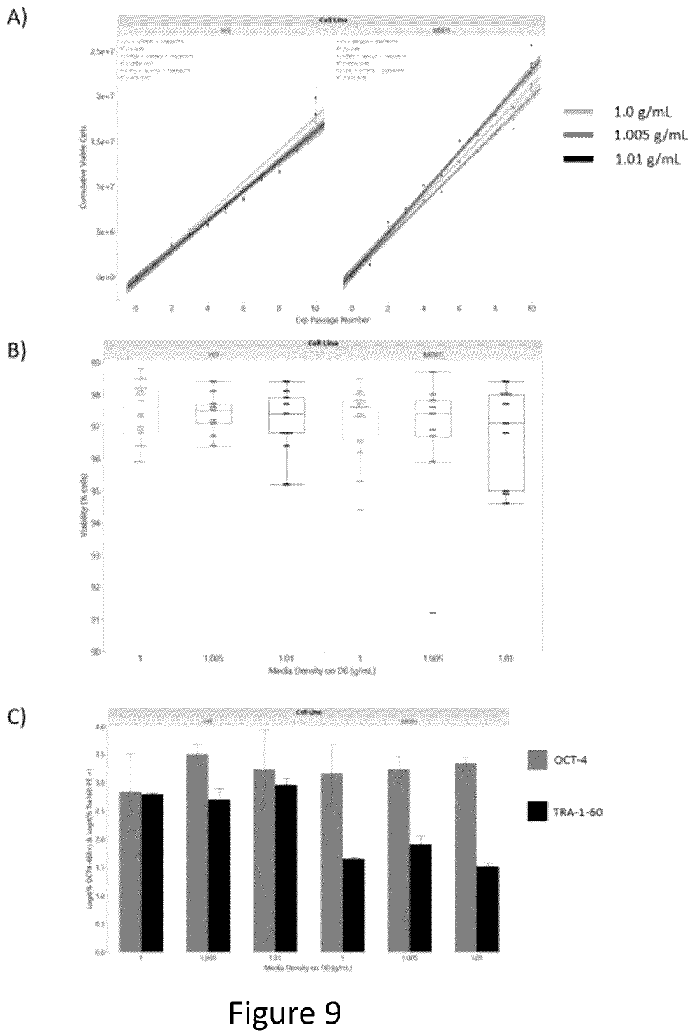

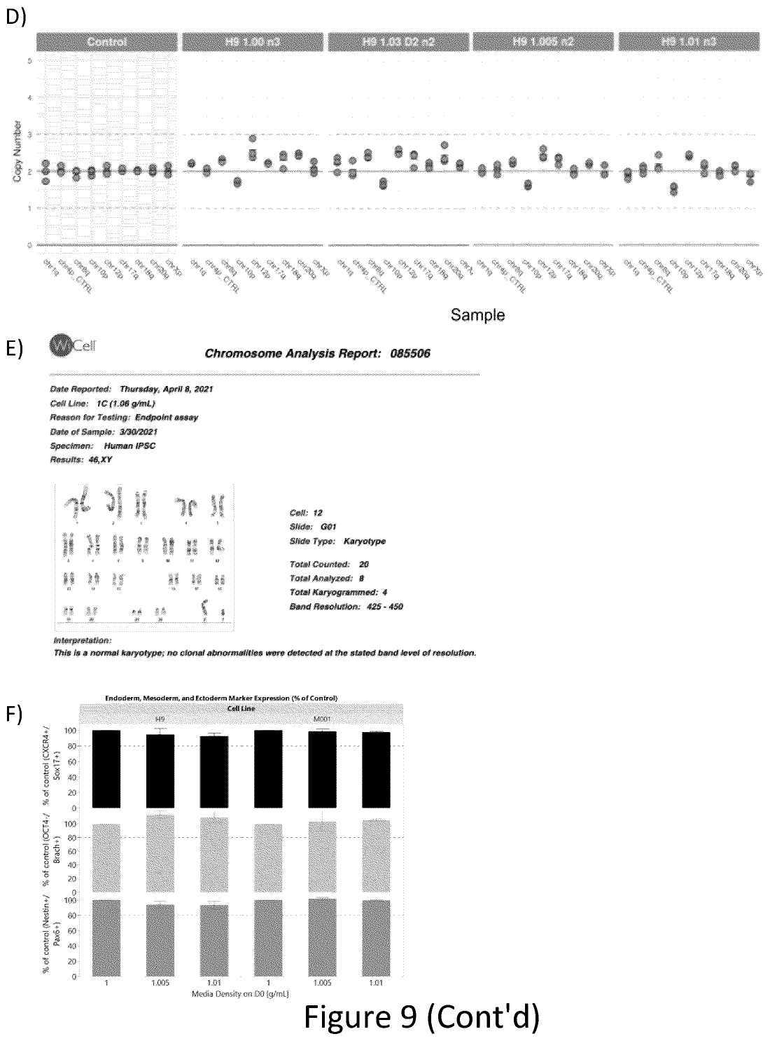

The cells cultured as described above for 10 passages were assessed for cumulative viable cells at each passage. On the day of passage, each of the 3 technical replicates per condition were exposed to ReLeSR (STEMCELL Technologies) for liberating colonies of undifferentiated PSC, and a resulting clump suspension was prepared by pooling the technical replicates of a condition. Viable cell counts for each pooled sample was determined using the Chemometec NucleoCounter NC-250™ (viability and cell counts obtained using NC-slides A2™ with Reagent A100 and B). Statistics were done using an Analysis of Means in JMP to compare the slope of each experimental condition to the group average. For H9 cells, no significant differences in cumulative viable cell yield between the control (1.00 g/mL) and experimental conditions (1.005 g/mL and 1.01 g/mL) were observed (FIG. 9A). For M001 cells, the 1.01 g/mL condition was significantly different from the control, but as the slope is greater than the control, this is not concerning (FIG. 9A).

Following the 10-passage experiment described above, the cells obtained in each condition were assessed for % viability (quantified as above using the NC-250). No statistical differences in percent viability between the control (1.00 g/mL) and experimental conditions (1.005 g/mL and 1.01 g/mL) were observed for either cell line following the 10-passage assay (FIG. 9B). The data is represented with an outlier box plot where the horizontal line in the box represents the median value. Stats were done using an ANOVA and post-hoc Tukey HSD test (all pairs) in JMP.

After culturing the cells for 10 passages, as described above, the expression of pluripotency markers was assessed among the different conditions tested. Cells were stained with either PE anti-human TRA-1-60-R antibody (BioLegend) or Alexa Fluor® 488 anti-Oct4 antibody (BioLegend), and assessed by flow cytometry (Guava viaCyte 12HT, Millipore). No significant differences in OCT-4 and TRA-1-60 expressions were observed among the different conditions tested, suggesting that punctuated exposure to density-adjusted media during passaging operations does not negatively affect the cumulative viable cell number, percent cell viability, and the undifferentiated phenotype of PSCs (FIG. 9C).

After culturing the cells for 10 passages, as described above, the accumulation of recurrent mutations and the karyotypes of the end point cells was assessed among the different conditions tested. To assess for the accumulation of common mutations known to arise among PSC, the genomic DNA was isolated from each sample cultured in the “Standard” density medium condition and in the indicated density-adjusted media conditions. The isolated DNA was analyzed using the Genetic Analysis Kit (STEMCELL Technologies) for 8 common karyotype abnormalities observed in PSCs and a control locus. The results of qPCR experiments using the QuantStudio 5 Real-Time PCR System (Applied Biosystems) are shown in FIG. 9D. The copy numbers at the assessed genomic regions do not appear to markedly vary for either the samples grown and passaged exclusively in Standard density medium or those that were passaged in density-adjusted media formulations. Samples were also sent for karyotype analysis by G-banding (WiCell) to confirm the results obtained using the Genetic Analysis Kit (STEMCELL Technologies). A sample Chromosome Analysis Report is shown in FIG. 9E, but the overall results of this investigation confirmed that culturing PSC with transient exposure (for about 48 hours) to density adjust media formulations over multiple passages does not affect the genetic stability of the cells (i.e. the cells are not more susceptible to an accumulation of rearrangements than cells cultured under control conditions).

After culturing the cells for 10 passages, as described above, the differentiation capacity was assessed among the different conditions tested. Cells were harvested and cultured to induce differentiation into the three embryonic germ layers using the STEMdiff Trilineage Differentiation Kit (STEMCELL Technologies). At the end of the culture period, cells were stained with either human SOX17 APC-conjugated antibody (R&D Systems) and human CXCR4 PE-conjugated antibody (R&D Systems) for the endoderm lineage, human/mouse Brachyury APC-conjugated antibody (R&D Systems) and Alexa Fluor® 488 anti-Oct4 antibody (BioLegend) for the mesoderm lineage, or Alexa Fluor 647 Mouse Anti-Human PAX-6 (BD Biosciences) and Anti-human Nestin AlexaFluor 488 (Thermo Fisher Scientific) for the ectoderm lineage, and assessed by flow cytometry (Guava viaCyte 12HT, Millipore). No significant differences in double stained marker expression were observed among the different conditions tested, and all conditions had comparable differentiation efficiency (within 80%) to cells maintained in a “Standard” density medium (FIG. 9F). The figure shows the mean (n=3) marker expression, with error bars showing standard deviation.

Overall, the results in this Example 7 show that in comparison to cells exposed to exclusively “Standard” density media, the repeated exposure of cells during passaging to density-adjusted media formulations (in particular during the aliquoting and seeding operations) does not appear to result in detrimental effects on cumulative viable cell number, percent cell viability, the expression of pluripotency markers, the accumulation of relatively larger scale genetic aberrations, and the ability of PSC to differentiate to the germ layers. Accordingly, density-adjusted media may be used in the processing of cells (e.g. to facilitate the culture, passaging, isolation/separation/enrichment, and sampling) without any apparent risk to the cells themselves.

### Example 8: Harvesting PSC Aggregates from the Wells of a Microwell Device Using Density-Adjusted Media

A single cell suspension of WLS-1C PSC was prepared as described in Example 1. Each well of an AggreWell 400 (STEMCELL Technologies) was seeded with 2 mL of a 1.2×10⁵ cells/mL suspension, such that each microwell of a well-received about 200 PSC. Briefly, AggreWell were prepared as recommended by the manufacturer and 1 mL of complete mTeSR™1 supplemented with 10 μM Rocki (Y-27632, STEMCELL Technologies) was added to each well thereof. Next, 1 mL of a 2.4×10 5 cells/mL single cell suspension of WLS-1C iPS cells in complete mTeSR™1 supplemented with 10 μM Rocki was added to the well to obtain a final concentration of 1.2×10⁵ cell/mL in each well. Aggregates were allowed to form for 2 days before harvesting the aggregates. Typically, harvesting of aggregates from a microwell device involves multiple rounds of removing and forcefully re-dispensing media to dislodge the aggregates.

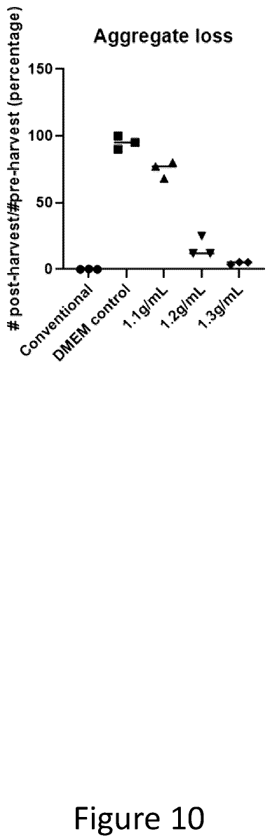

Here, on day 3 spent media was slowly removed and discarded. Different harvesting media were prepared as follows: control medium (1.0 g/mL), and media adjusted to either 1.1 g/mL, 1.2 g/mL, or 1.3 g/mL using iodixanol. After dispensing the various harvesting media into each well of the microwell device, aggregate loss was determined for each well. Using control harvesting medium, virtually all of the aggregates remained in the microwell device (FIG. 10, “DMEM control”). Progressively more aggregates could be recovered as the density of the harvesting medium increased (FIG. 10). A 1.3 g/mL harvesting medium recovered essentially all aggregates, similar to the conventional and time-consuming approach described above (FIG. 10).

### Example 9: In Process Use of Density-Adjusted Media

In automated processes for culturing PSC aggregates in suspension the recovery, passaging, and/or washing steps may be facilitated by flowing cell suspensions through a filter. Maintaining a homogeneous distribution of aggregates through use of density-adjusted liquids of this disclosure as they pass through the filter may prevent clogging.

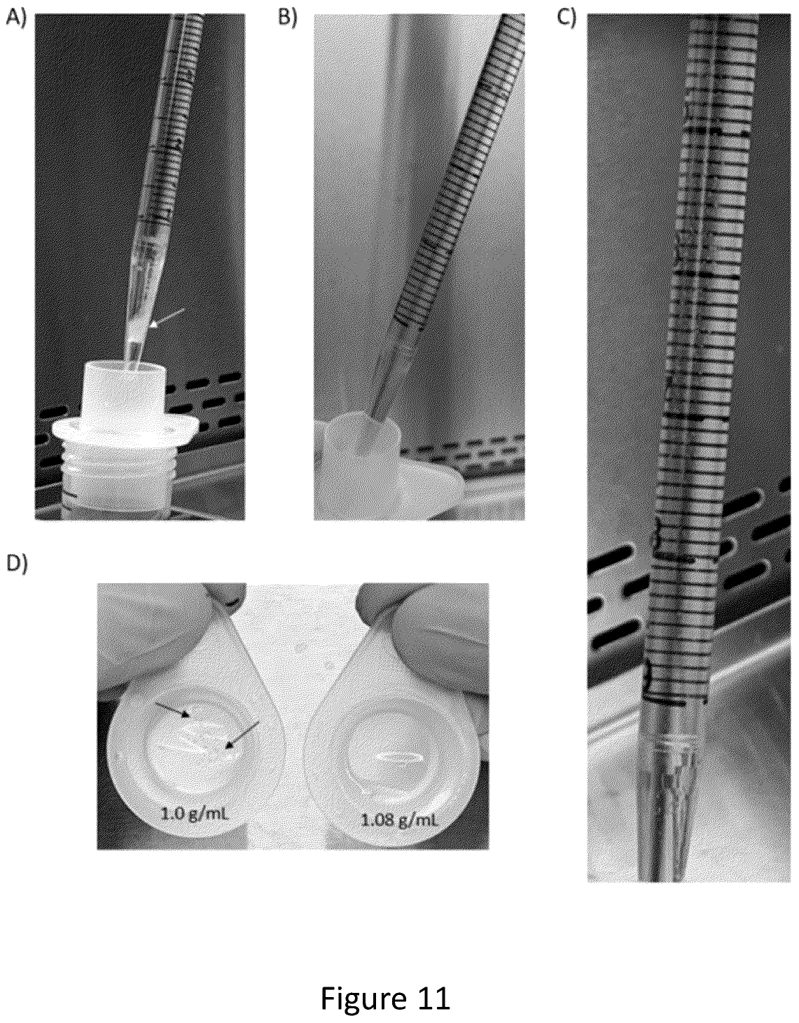

Aggregates of F016 cells of roughly 300-500 μm in diameter were suspended in either TeSR™ E8™ 3D medium (1 g/mL) or TeSR E8 (1.08 g/mL) and were taken up into a 10 mL transfer pipette. Within the volume/column of liquid in each transfer pipette, the aggregates were permitted to settle for about 20 to 30 seconds (FIGS. 11A, B, and C). Aggregates in standard medium (1.0 g/mL) rapidly settled within the volume/column of the liquid (FIG. 11A), but remained evenly distributed throughout the volume/column of a density-adjusted medium (1.08 g/mL) (FIGS. 11B, and FIG. 11C fora magnified view).

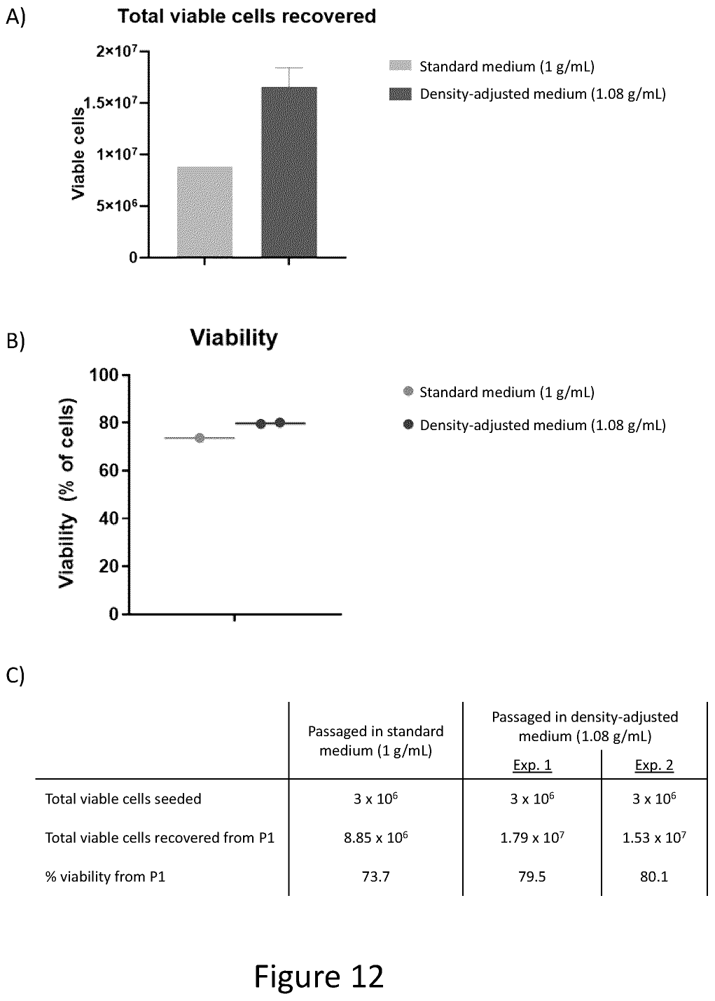

Each volume of medium in each pipette was passed through a 37 μm mesh filter and the residue on each filter was photographed (FIG. 11D). Only the condition where the aggregates were suspended in a standard culture medium (1.0 g/mL) resulted in a significant build-up of aggregates on the filter due to clogging. The filtrate from each condition was assessed for total viable cells recovered (FIG. 12A) and for % viability of recovered cells (FIG. 12B). As shown in FIGS. 12A and 12B, and in the summarized data in FIG. 12C, more cells are recovered following filter-based passaging of PSC aggregates when suspended (substantially homogeneously) in density-adjusted culture medium (1.08 g/mL) and such cells exhibit higher percent viability, in comparison to a settled population of aggregates in standard medium (1 g/mL).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of manipulating the localization of a population of particles, or one or more subpopulations thereof, within a volume of a liquid, comprising:
a) contacting the population of particles, or the one or more subpopulations thereof, with the liquid, the liquid supplemented with a specific gravity-modifying means and having a specific gravity that is not equal to unity; and
b) influencing buoyancy of the population of particles, or the one or more subpopulations thereof, within the volume of the liquid;
wherein the specific gravity of the liquid is adjusted either before or after contacting the population of particles, or the one or more subpopulations thereof, with the liquid, by varying a concentration of the specific gravity-modifying means in the liquid.

2. (canceled)

3. The method of claim 1, further comprising applying an energy input to the liquid to distribute the population or subpopulation(s) of particles throughout the volume of the liquid, optionally wherein the energy input is a turbulent mixing force.

4-5. (canceled)

6. The method of claim 3, further comprising deactivating the energy input, wherein distribution of the population or subpopulation(s) of particles is maintained throughout the volume of the liquid after deactivating the energy input.

7. (canceled)

8. The method of claim 6, further comprising removing a first aliquot of particles from the volume of the liquid, followed by removing a second aliquot of particles from the volume of the liquid, wherein a concentration of particles in the second aliquot is within about 80% or more of a concentration of particles in the first aliquot.

9. (canceled)

10. The method of claim 1, wherein an average density of the particles of the population, or the one or more subpopulations thereof, is the same as or within about +/−3% of the specific gravity of the liquid, and the population or subpopulation(s) is neutrally buoyant within the volume of the liquid.

11. The method of claim 1, further comprising biasing the population or subpopulation(s) of particles either upward from or downward toward a lower portion of the volume of the liquid, wherein
i) an average density of the particles of the population, or the one or more subpopulations thereof, is lower than the specific gravity of the liquid, and the population or subpopulation(s) is biased upward toward or to a gas-liquid interface of the volume of the liquid, or
ii) an average density of the particles of the population, or the one or more subpopulations thereof, is higher than the specific gravity of the liquid and the population or subpopulation(s) sediments within the volume of the liquid.

12. (canceled)

13. The method of claim 11, wherein the population or subpopulation(s) of particles is sedimented within the volume of the liquid prior to contacting the population or subpopulation(s) of particles with the liquid, and is biased upward within the volume of the liquid after coming into contact with the liquid.

14. (canceled)

15. The method of claim 1, wherein the specific gravity of the liquid is between about 1.0 g/mL and 1.5 g/m L.

16. The method of claim 1, wherein the specific gravity of the liquid is more than or less than +/−3% from unity.

17. The method of claim 1, wherein the specific gravity-modifying means is a specific gravity-modifying soluble solid or miscible solution.

18. The method of claim 17, wherein the specific gravity-modifying soluble solid is iodixanol, or the specific gravity-modifying miscible solution comprises iodixanol.

19-21. (canceled)

22. The method of claim 1, wherein the population or subpopulation(s) of particles is a population or subpopulation(s) of single cells or aggregates of cells.

23-27. (canceled)

28. The method of claim 27, further comprising culturing and/or expanding the population or subpopulation(s) of cells within the volume of the liquid, wherein the liquid is a cell culture medium.

29. (canceled)

30. A liquid for manipulating the localization of a population of particles, or one or more subpopulations thereof, the liquid comprising a diluent and a specific gravity-modifying means, wherein a specific gravity of the liquid is not equal to unity.

31. The liquid of claim 30, wherein the specific gravity-modifying means is a specific gravity-modifying soluble solid or miscible solution.

32. The liquid of claim 31, wherein the specific gravity-modifying soluble solid is iodixanol, or the specific gravity-modifying miscible solution comprises solubilized iodixanol.

33-36. (canceled)

37. The liquid of claim 30, wherein the diluent is a buffer, or a cell culture medium comprising one or more of a buffer, inorganic salts, trace elements, lipids, an albumin, growth factors, and cytokines.

38-39. (canceled)

40. The liquid of claim 30, wherein a viscosity of the liquid is within 10% of water.

41. The liquid of claim 40, wherein the viscosity is about 0.69 cP at 37° C.

42. The liquid of claim 30, wherein a specific gravity of the liquid is between about 1.0 g/mL and 1.5 g/m L.

43-44. (canceled)

45. The liquid of claim 30, wherein an average density of a population of particles, or of the one or more subpopulations thereof, in the liquid is
i) higher than the specific gravity of the liquid, or
ii) lower than the specific gravity of the liquid, or
iii) the same as or within +/−3% of the specific gravity of the liquid.

46-47. (canceled)
